(19) United States
(12) Patent Application Publication
(10) Pub. No.: US 2021/0378983 A1
(43) Pub. Date: Dec. 9, 2021

(54) Cannabinoids and Pearl Powder Topical

(71) Applicant: A&M Scientific, LLC, Huntsville, AL (US)

(72) Inventors: Matthew Rizzio, Huntsville, AL (US); Antonious Kurniawan, Madison, AL (US)

(73) Assignee: A&M Scientific, LLC, Huntsville, AL (US)

(21) Appl. No.: 16/892,550

(22) Filed: Jun. 4, 2020

(51) Int. Cl. A61K 31/05; A61K 35/618; A61K 9/107; A61K 47/44; A61K 47/34; A61K 47/02; A61K 47/10; A61K 9/00

(52) U.S. Cl. CPC A61K 31/05; A61K 35/618; A61K 9/107; A61K 9/0014; A61K 47/34; A61K 47/02; A61K 47/10; A61K 47/44

(57) ABSTRACT

The present disclosure is a substance for providing health benefits that is comprised of at least one cannabinoid and pearl powder.

### BACKGROUND

Cannabidiol (CBD) is a popular natural remedy that is used to treat common problems. CBD oil is one of over one hundred chemical compounds known as cannabinoids that are found in a marijuana plant. Unlike tetrahydrocannabinol (THC), which is the main psychoactive compound found in marijuana, CBD does not induce a high sensation when used, e.g., smoked or ingested. CBD may come in different forms, such as CBD oil, CBD isolate in crystals or powder, or CBD tinctures.

The CBD is known for having many health benefits. For example, CBD may be used to alleviate chronic pain by impacting the endocannabinoid receptor activity, thereby reducing inflammation and positively interacting with neurotransmitters. Additionally, CBD is known for reducing anxiety and depression, alleviating cancer-related symptoms, and reducing acne. CBD may also benefit heart health.

Pearl powder is also known as a natural remedy that is used to treat common problems. Pearl powder is made from freshwater pearls or saltwater pearls below jewelry grade. In one embodiment, the pearl powder can also come from high-quality jewelry pearls. The pearls are sterilized in boiling water and then milled into a fine powder using stainless steel grinding discs or by milling with small porcelain balls in moist conditions. The powder is sold as such or mixed into creams.

Pearl powder is known to have anti-inflammatory characteristics. It is also a detoxifying agent and relaxant. The benefits of pearl powder may come from the magnesium contained in the pearl powder. Thus, the pearl powder may also alleviate depression, anxiety, and sleep disorders. Further, the pearl powder can have anti-aging effects for wrinkles and skin care.

### DETAILED DESCRIPTION

The present disclosure is an exemplary cannabinoid and pearl powder topical. Topical means applied directly to a part of the body. In one embodiment, the topical is a cream. However, it may be made in other forms in other embodiments, e.g., a spray. The cannabinoid and pearl powder topical of the present disclosure comprises both cannabinoids and pearl powder. The cannabinoid mixed with the pearl power can exhibit many health benefits, which are described above.

To make the cannabinoid and pearl powder topical, the manufacturing process begins with hemp biomass. The hemp biomass refers to the harvested parts of a hemp plant, and the hemp biomass contains both cannabinoids and trace amounts of tetrahydrocannabinol (THC).

The hemp biomass is weighted and bagged. The type of bagged used is one that is capable of low temperatures. The bags may be micron nylon bags, for example.

Winterization is then induced. Winterization is the act of removing any unwanted fatty acids, plant materials, chlorophyll, cannabinoids, and terpenoids contained in the hemp biomass. This is the process in which non-polar substances (such as hash oil) are dissolved into a polar substance (most commonly ethanol) at sub-zero temperatures. This forces waxes and lipids (fatty acids) from the crude extract to the top of the mixture where they can be easily removed through the filtration process.

To winterize, the bagged hemp biomass is frozen to −60° C. The freezing of the bagged hemp biomass may be effectuated, for example, using cryogenic freeing. In this regard, use of a system employing liquid nitrogen could be used to freeze the bagged hemp biomass.

Once the bagged hemp biomass is frozen to −60° C. and winterized, the bag is inserted in a centrifuge and flooded with −60° C. Ethanol for Ethanol extraction. In this regard, the Ethanol is introduced to the bagged hemp biomass to extract the cannabinoids. Note that a centrifuge is a machine with a rapidly rotating container that applies centrifugal force to the bagged hemp biomass. The centrifugal force applied to the bagged hemp biomass separates desirable versus undesirable components. That is, the centrifugal force removes cannabis plant matter from the cannabinoid solution.

After the bagged hemp biomass has been spun in the centrifuge, the cannabinoid solution is filtered to remove undesirable components, such as the Ethanol. The cannabinoid solution is run a falling film evaporator. A falling film evaporator concentrates the cannabinoid solution. It uses evaporation and condensation under vacuum conditions to create the crude oil. In this stage, the Ethanol may be recouped and sent back through a closed loop system to a chilling tank to be used again

After the crude oil is obtained, any further Ethanol is extracted out of the crude oil. Decarboxylation is applied to the crude oil. Decarboxylation is the process in which cannabinoid(s) is activated. Before the extract is decarboxylated, there is no naturally occurring cannabinoids or THC present in the plant. Instead, these Phyto cannabinoids are found in an acidic state, better known as cannabidiolic acid (CBDA), tetrahydrocannabinolic acid (THCA) and cannabigerolic acid (CBGA). Other components found in the acidic state may be possible in other embodiments. The decarboxylation process converts these inactive components into active components giving you a crude oil product.

The crude oil product is than distilled. In one embodiment, short path distillation may be used. However, other types of distillation are possible in other embodiments.

The crude oil product is purified and processed creating a full spectrum distillate oil from the crude oil. Full spectrum distillate is the resulting product that contains all the cannabinoids found in the hemp plant.

The cannabinoid oil is further modified with pearl powder to create a cannabinoid pear powder topical. This may be effectuated by mixing the cannabinoid oil with pearl powder. In addition, the following ingredients may be combined to create the cannabinoid pearl powder topical. The top base comprises the following:
Emulsifying Agent
Cetearyl alcohol
Lanolin Oil
Silicone Oil
Hemp Extract—Distillate Oil

The bottom base comprises the following:
Water
Pearl Powder
Hydrogen Peroxide

In one embodiment, the cannabinoid and pearl powder topical is unscented. In another embodiment, the cannabinoid and pearl powder topical is scented using different fragrances.

Note that the distillate oil of the top base and the pearl powder of the bottom base are primary ingredients. In this regard, the other ingredients listed may be removed or modified or other ingredients may be added.

The cannabinoid and pearl powder topical can be used with Full Spectrum, Broad spectrum, or isolate. Further the cannabinoid and pearl powder topical may contain cannabidiol (CBD), Cannabigerol (CBG), cannabinol (CBN), etc. In another embodiment, the cannabinoid and pearl powder topical may contain CBDa, CBGa, CBNa, etc.

What we claim is:

1. A substance for providing health benefits, comprising:
at least one cannabinoid; and
pearl powder.

2. The substance of claim 1, further comprising an emulsifying agent.

3. The substance of claim 1, further comprising Cetearyl alcohol.

4. The substance of claim 1, further comprising lanolin.

5. The substance of claim 1, further comprising Silicone oil.

6. The substance of claim 1, further comprising water.

7. The substance of claim 1, further comprising hydrogen peroxide.
